(19) United States
(12) Patent Application Publication
(10) Pub. No.: US 2017/0189363 A1
(43) Pub. Date: Jul. 6, 2017

(54) GASTRIC ACID MODULATORS FOR ORAL DELIVERY OF PEPTIDES AND PROTEINS

(71) Applicant: NOVEN PHARMACEUTICALS, INC., Miami, FL (US)

(72) Inventors: Jeffrey Klein, Miami, FL (US); Anubhav Arora, Miami, FL (US); Puchun Liu, Miami, FL (US)

(73) Assignee: NOVEN PHARMACEUTICALS, INC., Miami, FL (US)

(21) Appl. No.: 15/393,497

(22) Filed: Dec. 29, 2016

Related U.S. Application Data
(60) Provisional application No. 62/272,941, filed on Dec. 30, 2015.

(51) Int. Cl. A61K 31/198; A61K 38/28; A61K 9/48; A61K 31/341

(52) U.S. Cl. CPC A61K 31/198; A61K 31/341; A61K 38/28; A61K 9/4808

(57) ABSTRACT

Described are methods for oral administration of therapeutic proteins and peptides, more specifically to the oral administration of therapeutic proteins and peptides with gastric acid modulators, and compositions of comprising a gastric acid modulator and a protein or peptide.

### CROSS-REFERENCE TO RELATED APPLICATIONS

This application claims the benefits under 35 U.S.C. §119(e) to U.S. provisional application 62/272,941, filed Dec. 30, 2015, the contents of which are incorporated herein by reference in their entirety.

### FIELD

The present disclosure generally relates to methods and compositions for oral administration of proteins or peptides using gastric acid modulators.

### BACKGROUND

Medical use of protein drugs is constrained by major drawbacks. Oftentimes, the short biological half-life of protein drugs requires frequent administration. Additionally, protein drugs may undergo rapid degradation in mucosal tissues and/or in the gastrointestinal tract. Such degradation limits the bioavailability and efficacy of orally administered protein drugs. Therefore, the most common mode of protein drug administration is by the parenteral route.

Parenteral administration suffers from its own drawbacks, such as significant inconvenience to patients, particularly for drugs that need to be administered daily, such as insulin. Moreover, parenteral administration requires parenteral delivery systems, adding to the costs of manufacturing, dispensing and administering the drugs. Although sophisticated non-parenteral pharmaceutical systems have been developed, such as intra-nasal systems, such systems also may suffer from low bioavailability, decreased compliance, and increased costs as compared to oral dosage forms. Thus, oral administration is generally preferred, having the major advantage of convenience and ease of use for increased patient compliance.

There is a need, therefore, for methods and compositions for orally administering protein drugs.

### SUMMARY

Described herein are methods of orally administering a protein or peptide to a subject in need thereof, comprising: (a) administering an amount of a gastric acid modulator to the subject in an amount effective to reduce levels of gastric acid in the stomach, and (b) orally administering the protein or peptide to the subject.

In some embodiments, the gastric acid modulator is selected from the group consisting of H₂ receptor blockers, proton pump inhibitors, prostaglandin E₁-like compounds, and antacids. In specific embodiments, the gastric acid modulator is an H₂ receptor blocker selected from the group consisting of cimetidine, ranitidine, famotidine, and nizatidine. In further specific embodiments, the gastric acid modulator is an H₂ receptor blocker and is administered at a dose of from about 1 mg to about 800 mg. In specific embodiments, the gastric acid modulator is a proton pump inhibitor selected from the group consisting of omeprazole, lansoprazole, dexlansoprazole, esomeprazole, pantoprazole rabeprazole, and ilaprazole. In further specific embodiments, the gastric acid modulator is a proton pump inhibitor and is administered at a dose of about 1 mg to about 150 mg. In specific embodiments, the gastric acid modulator is a prostaglandin E₁-like compound selected from the group consisting of enprostil, rioprostil, and misprostol. In further specific embodiments, the gastric acid modulator is a prostaglandin E₁-like compound and is administered at a dose of about 10 micrograms to about 1000 micrograms. In specific embodiments, the gastric acid modulator is an antacid selected from the group consisting of sodium bicarbonate, potassium bicarbonate, calcium carbonate, calcium bicarbonate, aluminum bicarbonate, aluminum hydroxide, magnesium bicarbonate, magnesium hydroxide, and magnesium trisilicate, and combinations thereof. In specific embodiments, the gastric acid modulator is an antacid and is administered at a dose of about 10 mg to about 8000 mg.

In any of these methods, the gastric acid modulator may be administered prior to orally administering the protein or peptide. In some embodiments, the gastric acid modulator may be administered orally. In some embodiments, the gastric acid modulator is administered substantially simultaneously with the protein or peptide in the same composition or in separate compositions. In some embodiments, the gastric acid modulator and the protein or peptide are orally administered in the same composition.

In any of these methods, the protein or peptide may be useful in the prevention, treatment or diagnosis of a disease or condition in the subject. In some embodiments, the described methods are effective to treat a disease or condition in the subject.

In some embodiments, the protein or peptide is selected from the group consisting of growth factors, cytokines, peptide hormones, analgesic peptides, enzymes, blood coagulating factors, peptide neurotransmitters, and antibodies, and diagnostically or therapeutically effective fragments thereof, and pharmaceutically acceptable salts thereof. In specific embodiments, the protein or peptide is selected from the group consisting of insulin, oxytocin, parathyroid hormone, heparin, calcitonin, vasopressin, and octreotide, and diagnostically or therapeutically effective fragments thereof, and pharmaceutically acceptable salts thereof.

In some embodiments, the amount of gastric acid modulator administered is effective to increase the pH in the stomach.

In some embodiments, the methods are effective to increase the oral bioavailability of the protein or peptide.

Also described herein are oral pharmaceutical compositions for oral administration of a therapeutic protein or peptide, comprising: (a) a gastric acid modulator in an amount effective to decrease gastric acid in the stomach, and (b) a therapeutically effective amount of a protein or peptide.

In some embodiments, the oral pharmaceutical composition further comprises an absorption promoter. In some embodiments, the absorption promoter comprises N-[8-(2-hydroxy-benzoyl)]aminocaprylic acid (SNAC). In some embodiments, the oral pharmaceutical composition is in the form of a powder, capsule, tablet, mucoadhesive patch, gastrointestinal patch, suspension, solution, emulsion, or syrup. In further specific embodiments, the oral pharmaceutical composition is in the form of a multilayer or capsule-in-capsule dosage form, wherein the gastric acid modulator is in an outer layer or outer capsule of the dosage form and the protein or peptide is in an inner layer or inner capsule of the dosage form.

In some embodiments, the oral pharmaceutical composition comprises a gastric acid modulator selected from the group consisting of H₂ receptor blockers, proton pump inhibitors, prostaglandin E₁-like compounds, and antacids. In some embodiments, the gastric acid modulator is an H₂ receptor blocker selected from the group consisting of cimetidine, ranitidine, famotidine, and nizatidine. In some embodiments, the gastric acid modulator is a proton pump inhibitor selected from the group consisting of omeprazole, lansoprazole, dexlansoprazole, esomeprazole, pantoprazole rabeprazole, and ilaprazole. In some embodiments, the gastric acid modulator is a prostaglandin E₁-like compound selected from the group consisting of enprostil, rioprostil, and misprostol. In some embodiments, the gastric acid modulator is an antacid selected from the group consisting of sodium bicarbonate, potassium bicarbonate, calcium carbonate, calcium bicarbonate, aluminum bicarbonate, aluminum hydroxide, magnesium bicarbonate, magnesium hydroxide, and magnesium trisilicate, and combinations thereof.

In some embodiments, the oral pharmaceutical composition comprises a protein or peptide useful in the prevention, treatment or diagnosis of a disease or condition in the subject. In some embodiments, the protein or peptide is selected from the group consisting of growth factors, cytokines, peptide hormones, analgesic peptides, enzymes, blood coagulating factors, peptide neurotransmitters, and antibodies, and diagnostically or therapeutically effective fragments thereof, and pharmaceutically acceptable salts thereof. In further specific embodiments, the protein or peptide is selected from the group consisting of insulin, oxytocin, parathyroid hormone, heparin, calcitonin, vasopressin, and octreotide, and diagnostically or therapeutically effective fragments thereof, and pharmaceutically acceptable salts thereof.

In some embodiments, the oral pharmaceutical composition comprises an amount of gastric acid modulator effective to increase the pH in the stomach.

### BRIEF DESCRIPTION OF THE DRAWINGS

FIG. 1A and FIG. 1B illustrate blood glucose levels (mg/dL) after administration of insulin in accordance with methods described herein (FIG. 1A) or without administration of a gastric acid modulator (FIG. 1B), as described in Example 1. The dotted line represents the lower limit of quantitation for the glucometer used (20 mg/dl).

FIG. 2A and FIG. 2B illustrate blood glucose levels (mg/dL) after administration of insulin in accordance with methods described herein (FIG. 2A) or without administration of a gastric acid modulator (FIG. 2B), as described in Example 2. The dotted line represents the lower limit of quantitation for the glucometer used (20 mg/dl).

FIG. 3 illustrates blood glucose levels (mg/dL) after administration of a combination composition as described herein comprising insulin and a gastric acid modulator (♦) as compared to treatment with a composition that did not include the gastric acid modulator (▪), as described in Example 3.

FIG. 4 illustrates plasma levels of drug (pg/mL) after administration of a combination composition as described herein comprising oxytocin () as compared to treatment without administration of a gastric acid modulator (▪), as described in Example 4.

### DETAILED DESCRIPTION

Described herein are methods and compositions for oral administration of proteins or peptides using gastric acid modulators. In some embodiments, the methods are effective to increase the oral bioavailability of the protein or peptide. In some embodiments, the methods are effective for treating a disease or condition treatable by the protein or peptide. In some embodiments, the compositions are oral pharmaceutical composition for oral administration of a therapeutic protein or peptide. Also described are methods for preparing and using such compositions.

Without being bound by theory, the inventors believe that the gastric acid modulator modifies the pH in the gastrointestinal tract (such as in the stomach) such that the protein or peptide can be absorbed from the gastrointestinal tract (e.g., from the stomach) prior to being degraded by the normally highly acidic environment of the gastrointestinal tract (including the stomach).

### Definitions

Technical and scientific terms used herein have the meanings commonly understood by one of ordinary skill in the art to which the present invention pertains, unless otherwise defined. Reference is made herein to various methodologies known to those of ordinary skill in the art. Publications and other materials setting forth such known methodologies to which reference is made are incorporated herein by reference in their entireties as though set forth in full. Any suitable materials and/or methods known to those of ordinary skill in the art can be utilized in carrying out the present invention. However, specific materials and methods are described. Materials, reagents and the like to which reference is made in the following description and examples are obtainable from commercial sources, unless otherwise noted.

As used herein, the singular forms “a,” “an,” and “the” designate both the singular and the plural, unless expressly stated to designate the singular only.

The term “about” means that the number comprehended is not limited to the exact number set forth, and refers to ranges substantially around the stated value while not departing from the scope of the invention. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein “subject” denotes any animal in need of protein or peptide therapy, including humans. For example, a subject may be suffering from or at risk of developing a condition that can be treated or prevented with a protein or peptide or may be taking a protein or peptide for health maintenance purposes, or for other purposes.

As used herein, the phrases “therapeutically effective amount” and “therapeutic level” mean that drug dosage or plasma concentration in a subject, respectively, that provides the specific pharmacological response for which the protein or peptide is administered to a subject in need of such treatment, for whatever reason. It is emphasized that a therapeutically effective amount or therapeutic level of a protein or peptide will not always be effective in treating the target disease or condition in a given subject, even though such amount is deemed to be a therapeutically effective amount by those of skill in the art. For illustration only, exemplary therapeutically effective amounts of exemplary proteins or peptides are provided below with reference to adult human subjects. Those skilled in the art can adjust such amounts in accordance with standard practices as needed to treat a specific subject and/or condition/disease.

While “peptide” and “protein” may have different meanings or connotations in the art, they are used interchangeably herein. Thus, any mention of a peptide applies equally to proteins, and vice versa. The terms embrace naturally occurring peptides and proteins, as well as peptides and proteins in which one or more amino acid residues is a non-naturally occurring amino acid or has been modified or conjugated to an additional moiety.

As used herein, “pharmaceutically acceptable salt” has the standard meaning in the art. Pharmaceutically acceptable salts are well known in the art and described in, for example, S. M. Berge et al., J. Pharmaceutical Sciences, 66: 1 (1977).

In the following description, details and specific embodiments are set forth in order to provide a thorough understanding of the invention, for purposes of explanation and not limitation. It will be apparent to those skilled in the art that the invention may be practiced in other embodiments that depart from the details and specific embodiments.

### Proteins and Peptides

As noted above, the present disclosure generally relates to methods and compositions for oral administration of proteins or peptides. The protein or peptide may be any protein or peptide, including any protein or peptide useful in the prevention, treatment or diagnosis of a disease or condition in the subject.

In some embodiments, the protein or peptide is selected from the group consisting of hormones, growth factors, cytokines, analgesic peptides, enzymes, blood coagulating factors, peptide neurotransmitters, and antibodies, and diagnostically or therapeutically effective fragments thereof, including pharmaceutically acceptable salts thereof.

In specific embodiments, the protein or peptide is selected from the group consisting of insulin, oxytocin, parathyroid hormone, heparin, calcitonin, vasopressin, and octreotide, and diagnostically or therapeutically effective fragments thereof, including pharmaceutically acceptable salts thereof. In further specific embodiments, the protein or peptide is insulin, or a therapeutically effective fragment thereof, including pharmaceutically acceptable salts thereof. In further specific embodiments, the protein or peptide is oxytocin, or a therapeutically effective fragment thereof, including pharmaceutically acceptable salts thereof. In further specific embodiments, the protein or peptide is parathyroid hormone, or a therapeutically effective fragment thereof, including pharmaceutically acceptable salts thereof. In a specific embodiment, the protein or peptide is parathyroid hormone fragment 1-34 (PTH 1-34), or a pharmaceutically acceptable salts thereof.

### Gastric Acid Modulators

As noted above, the present disclosure generally relates to methods and compositions that use a gastric acid modulator. The gastric acid modulator may be any compound able to modify the pH in the stomach upon parenteral or oral administration.

In some embodiments, the gastric acid modulator is selected from the group consisting of H₂ receptor blockers, proton pump inhibitors, prostaglandin E₁-like compounds, and antacids, including pharmaceutically acceptable salts thereof.

In specific embodiments, the gastric acid modulator is an H₂ receptor blocker selected from the group consisting of cimetidine, ranitidine, famotidine, nizatidine, and pharmaceutically acceptable salts thereof. In other specific embodiments, the gastric acid modulator is a proton pump inhibitor selected from the group consisting of omeprazole, lansoprazole, dexlansoprazole, esomeprazole, pantoprazole rabeprazole, ilaprazole, and pharmaceutically acceptable salts thereof. In further specific embodiments, the gastric acid modulator is a prostaglandin E₁-like compound selected from the group consisting of enprostil, rioprostil, misprostol, and pharmaceutically acceptable salts thereof. In specific embodiments, the gastric acid modulator is an antacid, such as a potassium-, sodium-, calcium-, aluminum-, or magnesium-based antacid, such as an antacid selected from the group consisting of sodium bicarbonate, potassium bicarbonate, calcium carbonate, calcium bicarbonate, aluminum bicarbonate, aluminum hydroxide, magnesium bicarbonate, magnesium hydroxide, magnesium trisilicate, and combinations thereof. In specific embodiments, the gastric acid modulator is ranitidine, or a pharmaceutically acceptable salt thereof, such as ranitidine hydrochloride.

In some embodiments, two or more gastric acid modulators are used. In specific embodiments, two or more gastric acid modulators having different mechanisms of action are used, such as an H₂ receptor blocker and a proton pump inhibitor, an H₂ receptor blocker and a prostaglandin E₁-like compound, a proton pump inhibitor and a prostaglandin E₁-like compound, an H₂ receptor blocker, a proton pump inhibitor, and a prostaglandin E₁-like compound, an H₂ receptor blocker and an antacid, a proton pump inhibitor and an antacid, a prostaglandin E₁-like compound and an antacid, an H₂ receptor blocker, proton pump inhibitor and antacid, an H₂ receptor blocker, prostaglandin E₁-like compound and antacid, a proton pump inhibitor, prostaglandin E₁-like compound and antacid, an H₂ receptor blocker, proton pump inhibitor and antacid, or an H₂ receptor blocker, a proton pump inhibitor, a prostaglandin E₁-like compound and antacid.

### Methods of Administration

Described herein are methods of orally administering a protein or peptide to a subject in need thereof, comprising (a) administering a gastric acid modulator to the subject, and (b) orally administering the protein or peptide to the subject. In some embodiments, the subject is a mammal, including domesticated mammals (e.g. cats and dogs), livestock/farm animals (e.g., horses and cows), and humans. In specific embodiments, the subject is a human.

In some embodiments, the methods described herein are effective to treat a disease or condition in the subject that is treatable by the protein or peptide. In accordance with such embodiments, the methods may comprise administering a therapeutically effective amount of the protein or peptide. Therapeutically effective amounts of protein and peptide drugs are known in the art, and vary with the protein or peptide drug being administered and the intended effect. For example, insulin may be administered in an amount effective to achieve control of blood glucose levels and/or treat diabetes, heparin may be administered in an amount effective to achieve a target blood clotting time and/or treat a blood clotting disorder; vasopressin may be administered in an amount effective to achieve a target level of serum electrolytes and/or treat cardiovascular disease, including heart disease, stroke, or thromboembolism, and calcitonin may be administered in an amount effective to achieve target blood calcium levels or amelioration of osteoporosis symptoms and/or treat hypercalcemia or osteoporosis. In some embodiments, the amount of protein or peptide required to constitute a therapeutically effective dose is less than the amount required in methods that do not include the administration of a gastric acid modulator.

In some embodiments, the methods comprise administering an amount of gastric acid modulator effective to reduce gastric acid secretions. In some embodiments, the gastric acid modulator is administered in an amount effective to reduce gastric acid levels in the gastrointestinal tract by at least about 5%, 10%, 20%, 30%, 40%, 50%, 60%, 70%, 80%, or 90%, or to effectively eliminate or neutralize gastric acid in the gastrointestinal tract. In some embodiments, the gastric acid modulator is administered in an amount effective to reduce gastric acid levels in the stomach by at least about 5%, 10%, 20%, 30%, 40%, 50%, 60%, 70%, 80%, or 90%, or to effectively eliminate or neutralize gastric acid in the stomach. In some embodiments, the gastric acid modulator is administered in an amount effective to reduce gastric acid levels in the gastrointestinal tract by 5%-100%. In some embodiments, the gastric acid modulator is administered in an amount effective to reduce gastric acid levels in the stomach by 5%-100%.

Amounts of gastric acid modulators effective to reduce gastric acid levels are known in the art. For example, an H₂ receptor blocker may be administered at doses of from about 1 mg to about 800 mg, including from about 5 mg to about 400 mg, such as about 25 mg, about 50 mg, about 75 mg, about 100 mg, about 125 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 750 mg, or about 800 mg; a proton pump inhibitor may be administered at doses of about 1 mg to about 150 mg, including from about 5 mg to about 100 mg, such as about 10 mg, about 20 mg, about 30 mg, about 40 mg, about 50 mg, about 60 mg, about 70 mg, about 80 mg, about 90 mg, or about 100 mg; a prostaglandin E₁-like compound may be administered at doses of about 10 micrograms to about 1000 micrograms, including from about 35 micrograms to about 600 micrograms, such as about 35 micrograms, about 50 micrograms, about 100 micrograms, about 200 micrograms, about 300 micrograms, about 400 micrograms, about 500 micrograms, or about 600 micrograms; and an antacid may be administered at doses of about 10 mg to about 8000 mg, including from about 50 mg to about 1000 mg, such as about 20 mg, about 50 mg, about 80 mg, about 100 mg, about 200 mg, about 400 mg, about 500 mg, about 1000 mg, or about 2000 mg.

In some embodiments, the methods comprise administering an amount of gastric acid modulator effective to increase the pH in the gastrointestinal tract, including an amount effective to increase the pH in the gastrointestinal tract to a level at which the protein or peptide is more stable. In some embodiments, the methods comprise administering an amount of gastric acid modulator effective to increase the pH in the stomach, including an amount effective to increase the pH in the stomach to a level at which the protein or peptide is more stable. In some embodiments, the methods comprise administering an amount of gastric acid modulator effective to increase the pH in the stomach to greater than 2, greater than 2.5, greater than 3, greater than 3.5, greater than 4, greater than 4.5, greater than 5, greater than 5.5, or greater than 6.

In some embodiments, the methods are effective to increase the oral bioavailability of the protein or peptide. In specific embodiments, the bioavailability of the protein or peptide administered according to the methods described herein is increased by about 10% or more, as compared to the bioavailability of the protein or peptide when administered without a gastric acid modulator.

The gastric acid modulator may be administered by any route of administration as long as it is effective to reduce gastric acid levels and/or increase the pH in the gastrointestinal tract (or the stomach) when administered by that route. Thus, the gastric acid modulator may be administered parenterally, including intravenously, or orally.

The protein or peptide is administered orally.

In some embodiments, the gastric acid modulator and protein or peptide are administered sequentially. For example, the gastric acid modulator may be administered about 15 minutes, about 30 minutes, about 1 hour, about 90 minutes, about 2 hours, or longer, prior to the administration of the protein or peptide. In other embodiments, the gastric acid modulator and protein or peptide are administered essentially simultaneously, from the same composition or from separate compositions, by the same or different routes of administration.

In some embodiments, the gastric acid modulator and protein or peptide are administered the same number of times per day, including one, two, three, four, or more times per day. In other embodiments, the gastric acid modulator and protein or peptide are administered a different number of times per day. For example, the gastric acid modulator may be administered once per day and the protein or peptide may be administered twice, three times, or more times, per day.

### Compositions

The gastric acid modulator may be formulated in any composition suitable for the intended route of administration. Compositions suitable for formulating and administering gastric acid modulators parenterally and orally are known in the art.

The protein or peptide may be formulated in any composition suitable for oral administration. Compositions suitable for formulating and administering proteins and peptides for oral administration are known in the art. In some embodiments, the proteins and peptides are formulated in a manner intended to protect the protein or peptide from the gastrointestinal tract, such as in an enteric coated composition or liposome formulation. In other embodiments, the proteins and peptides are not formulated in a manner intended to protect the protein or peptide from the gastrointestinal tract, e.g., they are not formulated in an enteric coated composition or within a liposome formulation.

In some embodiments, the gastric acid modulator and the protein or peptide are formulated in the same composition suitable for oral administration. Exemplary compositions for oral administration include, but are not limited to, solid, semi-solid, gel, paste, liquid, crystalline or encapsulated forms. Non-limiting examples of these forms include powders (including sachets), capsules, tablets, mucoadhesive patches, gastrointestinal patches, suspensions, solutions, emulsions and syrups.

In specific embodiments, the gastric acid modulator and the protein or peptide are formulated in a multilayer tablet dosage form. In specific embodiments, the protein or peptide is contained in an inner layer and the gastric acid modulator is contained in an outer layer. The multilayer tablet may optionally be provided with a coating, such as an enteric coating.

In specific embodiments, the gastric acid modulator and the protein or peptide are formulated in a capsule-in-capsule dosage form. In specific embodiments, the protein or peptide is contained in an inner capsule and the gastric acid modulator is contained in an outer capsule.

In specific embodiments, the gastric acid modulator and protein or peptide are comprised in separate granules filled into the same capsule, or mixed and/or compressed together into the same layer of a tablet.

In variations of any of these embodiments, the gastric acid modulator may be formulated in immediate release granules or controlled release granules, including delayed release or sustained release. Additionally or alternatively, the protein or peptide independently may be formulated in immediate release granules or controlled release granules, including delayed release or sustained release granules.

In specific embodiments, the protein or peptide is formulated with an absorption enhancer, such as N[-(2-hydroxy-benzoyl)]aminocaprylic acid (SNAC), 8-(N-2-hydroxy-5-chlorobenzoyl)-amino-caprylic acid (5-CNAC), or N(10-[2-hydroxybenzoyl]amino)decanoic acid (SNAD). Thus, in specific embodiments, a composition may comprise a gastric acid modulator, a protein or peptide, and an absorption enhancer such as SNAC.

In any embodiments, the compositions may include one or more pharmaceutically acceptable ingredients, such as carriers, excipients, surfactants, diluents, disintigrants, buffers and/or stabilizers, such as described, for example, in Remington's Pharmaceutical Sciences (Remington's Pharmaceutical Sciences, 20th ed., Mack Publishing Company, Easton, Pa., USA, 2000).

### EXAMPLES

### Example 1

### Intravenous Ranitidine Hydrochloride Plus Oral Insulin

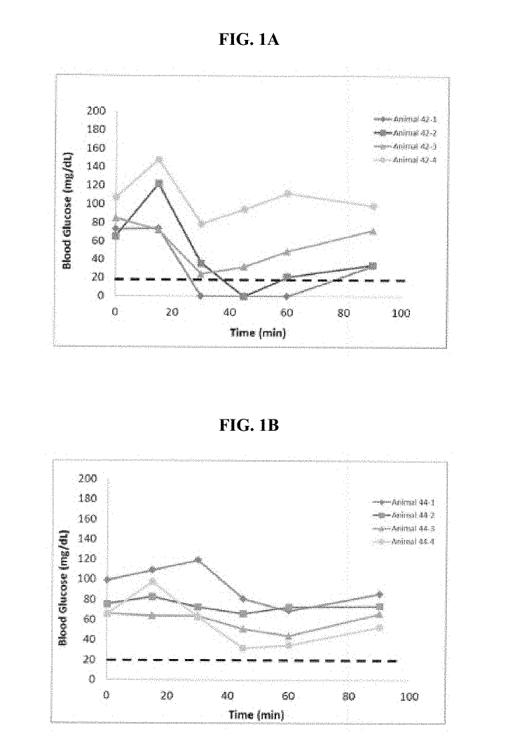

Rats were administered ranitidine hydrochloride (10 mg/kg) intravenously one hour prior to oral dosing with capsules comprising 0.3 mg insulin/20 mg SNAC. In vivo delivery of insulin was measured indirectly by assessing blood glucose levels at periodic intervals using a glucometer. Results are shown in FIG. 1A (each line represents one treated animal). For comparison, rats were orally administered capsules comprising 0.3 mg insulin/20 mg SNAC without administration of ranitidine hydrochloride, and blood glucose levels were assessed. Results are shown in FIG. 1B (each line represents one treated animal). As seen in the figures, blood glucose levels were lower after the protocol that included administration of ranitidine hydrochloride, indicating a greater bioavailability of insulin.

### Example 2

### Oral Ranitidine Hydrochloride Plus Oral Insulin

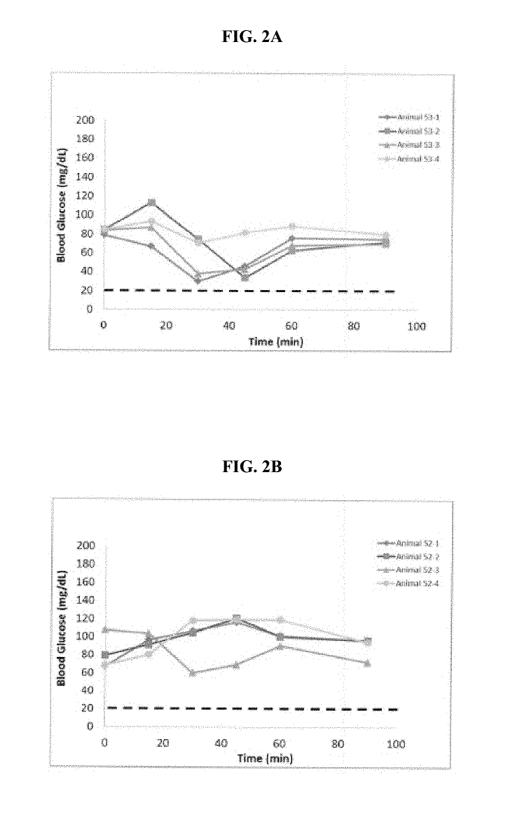

Rats were orally administered capsules comprising 0.3 mg insulin/10 mg SNAC/10 mg ranitidine hydrochloride, and blood glucose levels were measured at periodic intervals. Results are shown in FIG. 2A (each line represents one treated animal). For comparison, rats were orally administered capsules comprising 0.3 mg insulin/10 mg SNAC (i.e., without ranitidine hydrochloride), and blood glucose levels were assessed. Results are shown in FIG. 2B (each line represents one treated animal). As seen in the figures, blood glucose levels were lower after administration of the composition that included ranitidine hydrochloride, indicating a greater bioavailability of insulin.

### Example 3

### Capsule-In-Capsule Insulin and Ranitidine Hydrochloride

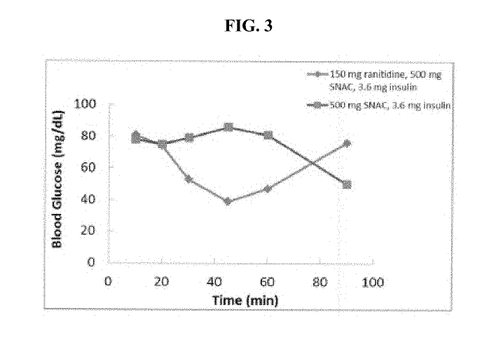

Capsule-in-capsule dosage forms were prepared having 3.6 mg insulin and 500 mg SNAC in an inner capsule and optionally having 150 mg ranitidine hydrochloride in an outer capsule. In particular, a size 0 capsule containing insulin and SNAC was placed inside an empty size 00 capsule, which was placed inside a size 000 capsule, optionally containing ranitidine hydrochloride. The capsules were administered to dogs and blood glucose levels were assessed. Results are shown in FIG. 3 (♦—with ranitidine hydrochloride; ▪—without ranitidine hydrochloride). As seen in the figures, blood glucose levels were lower after administration of the capsule-in-capsule dosage form that included ranitidine hydrochloride, indicating a greater bioavailability of insulin.

### Example 4

### Capsule-In-Capsule Insulin and Ranitidine Hydrochloride

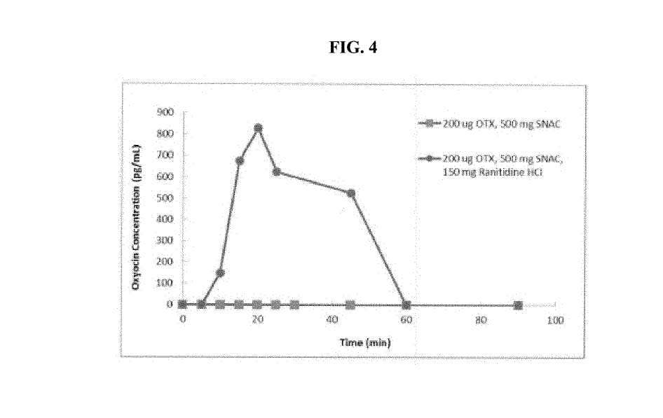

Capsule-in-capsule dosage forms were prepared having 200 μg oxytocin acetate and 500 mg SNAC in an inner capsule and optionally having 150 mg ranitidine hydrochloride in an outer capsule, similar to that described above in Example 3. The capsules were administered to dogs and plasma levels of oxytocin were assessed by liquid chromatography -mass spectrometry (LC-MS). Results are shown in FIG. 4 (—with ranitidine hydrochloride; ▪—without ranitidine hydrochloride). As seen in the figures, plasma levels of oxytocin were higher after administration of the capsule-in-capsule dosage form that included ranitidine hydrochloride, while plasma levels of oxytocin were undetectable after administration of the capsule-in-capsule dosage without ranitidine hydrochloride, indicating that oxytocin was absorbed through the gastrointestinal tract when administered with ranitidine hydrochloride, but was not absorbed through the gastrointestinal tract when administered without ranitidine hydrochloride.

What is claimed is:

1. A method of orally administering a protein or peptide to a subject in need thereof, comprising:
(a) administering an amount of a gastric acid modulator to the subject in an amount effective to decrease gastric acid levels in the stomach, and
(b) orally administering the protein or peptide to the subject.

2. The method of claim 1, wherein the gastric acid modulator is selected from the group consisting of H₂ receptor blockers, proton pump inhibitors, prostaglandin E₁-like compounds, and antacids.

3. The method of claim 1, wherein the gastric acid modulator is an H₂ receptor blocker selected from the group consisting of cimetidine, ranitidine, famotidine, and nizatidine.

4. The method of claim 1, wherein the gastric acid modulator is an H₂ receptor blocker and is administered at a dose of from about 1 mg to about 800 mg.

5. The method of claim 1, wherein the gastric acid modulator is a proton pump inhibitor selected from the group consisting of omeprazole, lansoprazole, dexlansoprazole, esomeprazole, pantoprazole rabeprazole, and ilaprazole.

6. The method of claim 1, wherein the gastric acid modulator is a proton pump inhibitor and is administered at a dose of about 1 mg to about 150 mg.

7. The method of claim 1, wherein the gastric acid modulator is a prostaglandin E₁-like compound selected from the group consisting of enprostil, rioprostil, and misprostol.

8. The method of claim 1, wherein the gastric acid modulator is a prostaglandin E₁-like compound and is administered at a dose of about 10 micrograms to about 1000 micrograms.

9. The method of claim 1, wherein the gastric acid modulator is an antacid selected from the group consisting of sodium bicarbonate, potassium bicarbonate, calcium carbonate, calcium bicarbonate, aluminum bicarbonate, aluminum hydroxide, magnesium bicarbonate, magnesium hydroxide, and magnesium trisilicate, and combinations thereof.

10. The method of claim 1, wherein the gastric acid modulator is an antacid and is administered at a dose of about 10 mg to about 8000 mg.

11. The method of claim 1, wherein the gastric acid modulator is administered prior to orally administering the protein or peptide.

12. The method of claim 1, wherein the gastric acid modulator is administered orally.

13. The method of claim 1, wherein the gastric acid modulator is administered substantially simultaneously with the protein or peptide in the same composition or in separate compositions.

14. The method of claim 1, wherein the gastric acid modulator and the protein or peptide are orally administered in the same composition.

15. The method of claim 1, wherein the protein or peptide is useful in the prevention, treatment or diagnosis of a disease or condition in the subject.

16. The method of claim 15, wherein the method is effective to treat a disease or condition in the subject.

17. The method of claim 1, wherein the protein or peptide is selected from the group consisting of growth factors, cytokines, peptide hormones, analgesic peptides, enzymes, blood coagulating factors, peptide neurotransmitters, and antibodies, and diagnostically or therapeutically effective fragments thereof, and pharmaceutically acceptable salts thereof.

18. The method of claim 1, wherein the protein or peptide is selected from the group consisting of insulin, oxytocin, parathyroid hormone, heparin, calcitonin, vasopressin, and octreotide, and diagnostically or therapeutically effective fragments thereof, and pharmaceutically acceptable salts thereof.

19. The method of claim 1, wherein the amount of gastric acid modulator administered is effective to increase the pH in the stomach.

20. The method of claim 1, wherein the method is effective to increase the oral bioavailability of the protein or peptide.

21. An oral pharmaceutical composition for oral administration of a therapeutic protein or peptide, comprising:
(a) a gastric acid modulator in an amount effective to decrease gastric acid levels in the stomach, and
(b) a therapeutically effective amount of a protein or peptide.

22. The composition of claim 21, further comprising an absorption promoter.

23. The composition of claim 22, wherein the absorption promoter comprises N-[8-(2-hydroxy-benzoyl)]aminocaprylic acid (SNAC).

24. The composition of claim 21, in the form of a powder, capsule, tablet, mucoadhesive patch, gastrointestinal patch, suspension, solution, emulsion, or syrup.

25. The composition of claim 21, in the form of a multilayer or capsule-in-capsule dosage form, wherein the gastric acid modulator is in an outer layer or outer capsule of the dosage form and the protein or peptide is in an inner layer or inner capsule of the dosage form.

26. The composition of claim 21, wherein the gastric acid modulator is selected from the group consisting of H₂ receptor blockers, proton pump inhibitors, prostaglandin E₁-like compounds, and antacids.

27. The composition of claim 21, wherein the gastric acid modulator is an H₂ receptor blocker selected from the group consisting of cimetidine, ranitidine, famotidine, and nizatidine.

28. The composition of claim 21, wherein the gastric acid modulator is a proton pump inhibitor selected from the group consisting of omeprazole, lansoprazole, dexlansoprazole, esomeprazole, pantoprazole rabeprazole, and ilaprazole.

29. The composition of claim 21, wherein the gastric acid modulator is a prostaglandin E₁-like compound selected from the group consisting of enprostil, rioprostil, and misprostol.

30. The composition of claim 21, wherein the gastric acid modulator is an antacid selected from the group consisting of of sodium bicarbonate, potassium bicarbonate, calcium carbonate, calcium bicarbonate, aluminum bicarbonate, aluminum hydroxide, magnesium bicarbonate, magnesium hydroxide, and magnesium trisilicate, and combinations thereof.

31. The composition of claim 21, wherein the protein or peptide is useful in the prevention, treatment or diagnosis of a disease or condition in the subject.

32. The composition of claim 31, wherein the protein or peptide is selected from the group consisting of growth factors, cytokines, peptide hormones, analgesic peptides, enzymes, blood coagulating factors, peptide neurotransmitters, and antibodies, and diagnostically or therapeutically effective fragments thereof, and pharmaceutically acceptable salts thereof.

33. The composition of claim 31, wherein the protein or peptide is selected from the group consisting of insulin, oxytocin, parathyroid hormone, heparin, calcitonin, vasopressin, and octreotide, and diagnostically or therapeutically effective fragments thereof, and pharmaceutically acceptable salts thereof.

34. The composition of claim 21, comprising an amount of gastric acid modulator administered effective to increase the pH in the stomach.
